(19) United States
(12) Patent Application Publication
(10) Pub. No.: US 2005/0152950 A1
(43) Pub. Date: Jul. 14, 2005

(54) Method and apparatus for macromolecular delivery using a coated membrane

(76) Inventor: Bruce N. Saffran, Brookline, MA (US)

Correspondence Address: DICKSTEIN SHAPIRO MORIN & OSHINSKY LLP, 2101 L Street, NW, Washington, DC, 20037, US

(21) Appl. No.: 10/990,535

(22) Filed: Nov. 18, 2004

Related U.S. Application Data
(63) Continuation of application No. 08/918,217, filed on Aug. 25, 1997, now abandoned.
(63) Continuation-in-part of application No. 08/557,423, filed on Nov. 13, 1995, now abandoned.

(51) Int. Cl. A61F 2/02; A61F 2/06

(52) U.S. Cl. 424/424; 623/1.42

(57) ABSTRACT

This invention relates to the delivery of medicines, macromolecules, or other treating materials to tissues and/or fluids that are to be injected or placed within a human or animal body. The invention describes a method of introducing a treating material to fluids ex corpora using a malleable fracture stabilization device with micropores for directed drug delivery (U.S. Pat. No. 5,466,262) into which a medicine has been incorporated, an apparatus for managing macromolecular distribution (U.S. Pat. No. 5,653,760) that has been coated with a treating material, or any surface to which has been affixed a treating material. The invention describes the use of a disposable housing that contains a semipermeable membrane to which a treating material has been affixed. The invention teaches the use of a dialysis membrane to which heparin or other anticoagulant has been affixed, thereby substantially preventing thrombosis on the membrane while limiting the amount of heparin that must be given systemically. Furthermore, the present invention provides a new and useful mechanism to deliver a treating material directly into the intravenous line from a pre-labeled vial, at a precise rate, and in a minimum volume of fluid. This invention can also be used to deliver treating materials directly to cells and tissues at a defined rate, while at the same time permitting small metabolites and other small toxins to wash away.

### BACKGROUND OF THE INVENTION

1. Field of Invention

This invention relates to the delivery of medicines, macromolecules, or other treating materials to tissues and/or fluids that are to be injected or placed within a human or animal body. The invention describes a method for introducing a treating material to fluids ex corpora using a malleable fracture stabilization device with micropores for directed drug delivery (U.S. Pat. No. 5,466,262) into which a medicine has been incorporated, an apparatus for managing macromolecular distribution (U.S. Pat. No. 5,653,760) that has been coated with a treating material, or any surface to which has been affixed a treating material. The invention also describes a disposable housing that contains a semipermeable membrane to which a treating material has been affixed. Although I will initially discuss this invention in the treatment of blood products during renal dialysis, I will also describe this invention as a method and apparatus to administer a treating material to intravenous fluids, and to the surface of intravenous bags, test tubes and tissue culture plates.

2. Description of the Prior Art

The seemingly simple process of administering medicines to patients in the hospital is deceptively complex. The medicines must be reconstituted correctly, and given at the appropriate times. Furthermore, many medicines require a precise rate of delivery in order to work optimally. All of these steps require nurses or other medical personnel to be ever vigilant to avert mistakes. Unfortunately, medicines do get injected too fast, or in the wrong dose. This is not a new problem, and many investigators have designed many different systems to simplify medicine delivery. The three most common settings in which precise intravenous medicine dosing is required are in renal dialysis, on the hospital inpatient service, and in cancer chemotherapy. I will discuss the present invention in these situations, and then discuss other uses of the present invention, both clinically and in the laboratory.

The goals of renal dialysis are simple: Pass a patient's blood across a semipermeable dialysis membrane, and the elevated concentration of electrolytes and other toxins in the blood will move passively across a concentration gradient out of the blood and into waste water. Although renal dialysis is an exceedingly common practice, the problems associated with handling dialysis blood and fluids during the filtration process are legion. Firstly, sterility must be maintained. If the solutions are not sterile or if there is no means to prevent the consequences of an infection when it does occur, the patient can become septic. If a patient does show signs of systemic infection during dialysis, intravenous antibiotics are given; however, because antibiotics take time to work, it would be desirable to bind endotoxins and other toxins as they are produced. Although there is an injectable form of anti-endotoxin antibody available, the drug is prohibitively expensive, has some undesirable side effects, and must be administered at just the right time for it to be efficacious. It would be highly desirable to have a way to bind these toxins as the blood passed over the dialysis membrane, thereby substantially lowering the chances of sepsis.

Secondly, when blood is passed outside of the body it has a tendency to clot. Blood clots, both large and small, can cause severe problems when they enter the systemic circulation. The clots usually form on the surface of the tubing and on the dialysis membrane. To thwart this problem, patients are usually given systemic anticoagulation. Systemic heparinization, however, has its own set of potential complications. Hemorrhagic stroke and internal bleeding, although uncommon, do occur. Diabetic patients are prone to retinal hemorrhages. Furthermore, because patients can also develop hematological abnormalities from heparin, it is desirable to use as little systemic heparin as possible.

Another setting in which intravenous medicines must be correctly mixed and administered is on the inpatient unit of a hospital. Because most medicines are clear solutions after they are reconstituted, one must take it on faith that the proper amount of the correct medicine is in the solution prior to giving it to the patient. Furthermore, once the medicine is administered to the patient, it must run in at a prescribed rate. Several ingenious devices have been developed to address the mixing and labeling problem. Perhaps the most successful of these involves the “spiking” of a labeled vial directly into the bag and leaving it attached to the bag. For medications insensitive to the rate of infusion, this system works well. Unfortunately, some medicines also require a reasonably precise rate of administration, e.g., the antibiotics vancomicin and erythromycin, and some cardiac medicines. Currently these limitations are addressed by using a mechanical pump, or diluting the medicines in a large volume of fluid. The latter technique becomes problematic when a patient with heart failure cannot tolerate the fluid load needed to get the medicine in. It would be very desirable to have a way to deliver medicine directly into the intravenous line from a pre-labeled vial, at a precise rate, and in a minimum volume of fluid.

Perhaps no setting requires more meticulous care of medication dosing and rate control than does the oncology service. Cancer chemotherapy drugs are given in very small precisely measured doses. If they are injected too fast, the local side effects can be severe. If a mistake is made in the pharmacy and too much drug or the wrong drug is in the syringe, disaster can result. Even in 1997, bags and syringes come up with hand written labels stating drug and dosage. The oncologist and the nurse must take on faith that the hand written label is correct. It would be very desirable to administer pre-loaded cartridges, designed to release medicine at a predetermined rate that are machine-labeled from the factory.

From the above discussion, it is clear that what is needed is a way to administer a known quantity of a known medicine, at a defined release rate. It is also clear that a mechanism is needed to bind undesirable molecules, e.g., bacterial endotoxins, as they come in contact with extra corporal blood.

It is an object of the present invention to provide and teach the use of an apparatus and method to bind such toxins at the surface of the membrane, thereby minimizing the chance that full-blown sepsis or other complications will occur.

It is a further object of the present invention to provide and teach the use of a dialysis membrane to which heparin or other anticoagulant has been affixed, thereby substantially preventing thrombosis on the membrane while limiting the amount of heparin that must be given systemically.

It is a further object of the present invention to provide a mechanism to deliver a treating material directly into the intravenous line from a pre-labeled vial, at a precise rate, and in a minimum volume of fluid.

I have also found unexpectedly that the invention can be used to deliver treating materials directly to cells, and tissues at a defined rate, while at the same time permitting small metabolites and other small toxins to wash away.

### SUMMARY OF THE INVENTION

The invention is a unique method of administering medicines or other treating materials directly and specifically to fluids or tissues on one side of a non-porous or semipermeable membrane. I have found unexpectedly that a malleable fracture stabilization device for directed drug delivery (U.S. Pat. No. 5,466,262) and an Apparatus for managing macromolecular distribution (U.S. Pat. No. 5,653,760) can also be used to deliver medicines to tissues and fluids outside of the body. Although the controlled-release properties of these two devices have made them ideally suited for local drug delivery from intravascular stents, catheters, coils, and balloons inside the body, they often cannot be modified after they are deployed. This reality makes it difficult to refill their medicine stores when the concentration of an affixed treating material falls too low. What is needed is an easy method of replenishing a medication supply while maintaining the drug delivery characteristics of the original devices. All embodiments of the present invention make use of non-porous or semipermeable membranes that have been coated with a treating material. These membranes can be supported by a semi-rigid or rigid scaffolding when it is required for optimal deployment of the membrane surface. These membranes, and the method of treating material attachment have been described in detail in my pending U.S. patent application Ser. No. 08/557423, and my issued patents, U.S. Pat. Nos. 5,466,262 and 5,653,760.

A principal embodiment of the present invention teaches the use of a semipermable membrane, which has been coated on one side with a medicine, to simultaneously filter blood or plasma and provide a medicine to either the concentrated solution or the filtrate. The semipermeable membrane can be the “minimally-porous” layer previously described in pending U.S. patent application Ser. No. 08/557423, U.S. Pat. Nos. 5,466,262 and 5,653,760, or any other semipermeable device. The membrane can be affixed to a rigid scaffold and placed in a cartridge. The membrane can also be placed within a cartridge without a scaffold. The cartridge is then positioned in series with standard dialysis or plasmaphoresis equipment. As a solution is dialyzed/phoresed the solution is also treated. It is not necessary for a treating material to be released into solution, rather it may also be used at the membrane interface, serve as a binding site for macromolecules, or serve a catalytic function while affixed to the membrane. When treating material stores get low or binding sited are used up, the cartridge can be replaced.

Another embodiment of the invention is the use of a non-porous or semipermeable membrane as a controlled way to introduce medicines to intravenous lines or other intravenous access sites. In this case, cartridges with a membrane containing a fixed amount of treating material are plugged into special receptacles of IV tubing. Currently, when patients in the hospital are in need of IV medicine, a nurse or pharmacist in the hospital adds medications to IV solutions at the time of use. These medicines are either “pushed” as a bolus or injected through a needle into an IV bag and hung over the patient's bed. Both of these methods require precise measurement by a trained professional at the time of need. Some medicines also require a reasonably precise rate of administration, e.g., the antibiotics vancomicin and erythromycin, and some cardiac medicines. The invention provides a semipermeable membrane with a treating material already affixed that is released at a predefined rate (see U.S. Pat. Nos. 5,466,262 and 5,653,760). Medicine is reconstituted as the fluid passes through the cartridge, and is released according to the nature of the bond between the membrane and treating material. This invention eliminates the need for measurement (cartridges would be pre-labeled with the brand name and amount of medicine that has already been affixed), eliminates the handling of needles to mix the drugs (fewer needle-sticks to staff), and eliminates the variability associated with different people mixing the medicines.

Another embodiment of the invention involves the use of a semipermeable membrane as a surface on which artificial skin or other cells can grow. In this embodiment, cells are plated onto a medication-coated membrane. Nutrients can be provided on the surface of the cells, and be restrained in the culture fluid around the cells by the semipermeable membrane. Cellular waste products and other small metabolites, however, can diffuse through the membrane and be washed away. The selective, rapid removal of waste products from the system drives cellular reactions forward. This effect markedly improves the time and efficiency of producing confluent cellular layers. In a similar fashion, a medication-coated membrane can be used to line the surface of disposable chambers for use in laboratory. Diagnostic evaluations of cells obtained at bone marrow aspiration or flow cytometry could be performed, as reagents could be selectively concentrated or removed, based on the composition of the membrane used. Treatment regimens of cells could also be undertaken in vitro using this system. Depending on the medicine affixed to the membrane, e.g., a particular cytotoxic drug, clonal selection or other selective cell proliferation treatment could be performed.

### BRIEF DESCRIPTION OF DRAWINGS

FIG. 1a is a side-view representation of a malleable fracture-stabilization device with micropores for directed drug delivery. The structure of this device is described in detail as part of pending U.S. patent application Ser. No. 08/557,423 and U.S. Pat. No. 5,466,262. Several methods for its use within the body are also described in those documents. Briefly, this is a two-layered device, the first layer is a minimally porous membrane (1), and the second layer is a microporous layer into which at least one treating material has been affixed (2). The treating material cannot pass through the minimally porous layer in significant amounts. Consequently, the treating material is released from the microporous layer only on the side opposite to the side to which the minimally porous layer has been affixed. The medicine originates within the pores of the microporous layer (5) and is released into solution (3) according to the properties of the microporous layer. An empty micropore is also shown (4). When the microporous layer is placed adjacent to the tissue to be treated, a medicine is directed preferentially toward that tissue. Surprisingly, this device can be used to address several problems associated with treating bodily fluids while they are outside of the body. In the attached text and drawings, I describe ways that a device with the same structure can be used to treat tissues and fluids outside of the body.

FIG. 1b is a representation of an apparatus for managing macromoleucular distribution (U.S. Pat. No. 5,653,760). This device is a modification of a malleable fracture-stabilization device with micropores for directed drug delivery, and can also be used to treat fluids and tissues extracorporally. This device is also constructed with a semipermeable membrane (1), but has a treating material attached directly to its surface either mechanically, or by chemical bond (6). Treating material released in solution is also depicted (3).

FIG. 2a depicts a rigid or semi rigid scaffold (7) onto which either a malleable fracture-stabilization device for directed drug delivery, an apparatus for managing macromolecular distribution, or other medication-coated membrane can be attached.

FIGS. 2b, 2c illustrate that the membrane (1) can be affixed to the outer surface of the scaffold (7) and an affixed treating material (6) directed toward the outer surface. The lumen of the tube (8) is also shown. FIG. 2b is a frontal view, and FIG. 2c is a cross section view.

FIGS. 2d, 2e illustrate that the membrane (1) can be affixed to the inner surface of the scaffold (7), and a treating material (6) can be directed toward the lumen (8). Treating material released into the lumen is shown (3). FIG. 2d is a frontal view, and FIG. 2e is a cross sectional view.

FIG. 2f depicts the two layered malleable fracture stabilization device with micropores for directed drug delivery affixed to a scaffold. The minimally porous layer (1) is affixed to the microporous layer (2). Note that a treating material (3) is directed specifically toward the lumen (8). Micropores containing medicine (5) and empty (4) are also shown.

FIG. 3a illustrates the membrane-covered scaffold within a housing (9). In this case, the device has been affixed to the inner surface of the scaffold (7), and the treating material is directed inward toward the lumen (8). Note the Luer adapter on either end of the cartridge (10). Other adapters, e.g., puncture adapters, could be used in place of the Luer adapters. In this arrangement, the scaffold and the outer surface of the semipermeable membrane are intimately associated with the housing such that no fluid can pass through the cartridge without passing through the lumen (8).

FIG. 3b shows the cartridge placed in series with intravenous line tubing. The afferent tubing (25), the efferent tubing (13) and the locking connectors (11) are shown for orientation. In this embodiment, the afferent fluid (12) would run through the lumen (8) of the device-filled cartridge. As the fluid passes through the cartridge (26), the treating material affixed to the membrane (6) would reconstitute and be released into solution (3). The soluble treating material within the efferent fluid (14) will subsequently travel away from the cartridge in the efferent stream (27). Again, the scaffold (7) and the outer surface of the semipermeable membrane (1) are intimately associated with the housing such that no fluid can pass through the cartridge without passing through the lumen.

FIG. 3c demonstrates a modification of the invention that permits one to take full advantage of the semipermeable nature of the medication-coated membrane. In this embodiment, there are two separate chambers, separated by the medication-coated semipermeable membrane. There is an enclosed chamber (23) in communication with the uncoated surface of the semipermeable membrane (1). The entrance and exit of this chamber is labeled “B”. The entrance to the lumenal chamber (8) is labeled “A”. The lumenal chamber needs to be separated by the minimally porous membrane (1) along its entire length, or bounded by a non-permeable septation (24) in the short segment connecting it to the housing (9).

FIG. 3d illustrates this embodiment in cross section. The two chambers, “A” and “B”, are separated by the medication-coated semipermeable membrane.

FIG. 3e depicts the two-chambered cartridge in use. In this example, two columns of fluid are entering the device. The first column is represented by the wavy arrows and enters from the lumenal entry port (12). The second column of fluid is represented by the straight arrows and enters via the external chamber entry port (15). The macromolecules originating within the lumenal fluid (12) are contained within the lumenal chamber (8) by the semipermeable membrane. The treating material is also contained within the lumenal chamber. If the side of the membrane containing the medication coating is placed facing the external chamber, the treating material remains in the external chamber. Small molecules (19), however, are free to cross the semipermeable membrane in response to changes in fluid composition. The fluid exiting the external chamber “B” (28), is similar to dialysis fluid in that its composition is a reflection of the molecular exchange that has taken place across the semipermeable membrane within the cartridge.

FIG. 3f is a cross section of FIG. 3e. Note that the free treating material is contained within the lumen, while small molecules are free to follow their concentration gradients. The two chambers, “A” and “B”, are separated by the medication-coated semipermeable membrane.

FIG. 4a depicts the medication-coated membrane as a surface upon which cells can be grown. In this figure, cells or cellular material have yet to be plated onto the membrane (1). The affixed treating material (6) and a sub-micron sized pore (18) is shown. In this case, the pore size would likely be approximately 200 Daltons, i.e., large enough to permit free passage of small metabolites (19), yet small enough to restrict passage of macromolecular nutrients (21), and of soluble treating material (3) (Please see FIG. 4b). In this example, the membrane has been supported over the floor of the culture plate by a scaffolding (29).

FIG. 4b depicts a medication-coated membrane (19) onto which cells (22) have been grown. In this case, some of the pre-affixed medicine (6) has become free in solution (3). Also note that macromolecular nutrients (21), and cellular waste products (19) are present in solution. A critical feature of this invention is the semipermeable nature of the membrane. Note that nutrients (21) and the free medicine (3) are contained next to the cells, whereas the small cellular waste products (1) are free to move through the pores (18). Since small metabolites are free to pass though the membrane, they can be washed away by a current of fluid (20). The scaffolding used to support the membrane off the floor of the dish is also shown (29).

### REFERENCE NUMERALS

1) Minimally porous membrane.

2) Microporous, medication-coating component of a malleable fracture stabilization device.

3) Treating material free in solution.

4) Empty micropore within the microporous, medication-containing component.

5) Medication-containing micropore within the microporous component.

6) Treating material affixed to the minimally porous membrane.

7) Scaffolding for a medication-coated membrane.

8) Lumen of a cartridge containing a medication-coated membrane.

9) Housing of cartridge.

10) Luer screw adapter receptacle.

11) Luer adapter of tubing.

12) Flow of fluid within the afferent tubing.

13) Wall of efferent tubing.

14) Eluted treating material within efferent tubing.

15) Fluid entering the external chamber of the two chambered cartridge.

18) Sub-micron sized pore in the semipermeable membrane.

19) Small cellular waste product.

20) Flow of wash fluid.

21) Macromolecular nutrient.

22) Cell.

23) Extralumenal chamber.

24) Non-porous septation affixing semipermeable membrane to cartridge housing.

25) Wall of afferent tubing.

26) Fluid flow in the cartridge lumen.

27) Fluid flow in the efferent tubing.

28) Fluid exiting the external chamber of the two chambered cartridge.

29) Scaffolding supporting semipermeable membrane above culture plate floor.

### DETAILED DESCRIPTION OF THE INVENTION

The invention consists of a devise and a method to administer a treating material in a directional fashion to tissues and fluids outside of human or animal bodies. I have found, unexpectedly, that my previous inventions, a malleable fracture stabilization device with micropores for directed drug delivery (Ser. No. 08/557,432, and U.S. Pat. No. 5,466,262), and my apparatus for managing macromolecular distribution (U.S. Pat. No. 5,653,760), have new and useful properties when used to treat tissues and fluids outside the body. The device of the present invention, and the method of its use, both involve the use of a semi-permeable membrane (1) to which has been affixed at least one treating material (3) which cannot pass through the membrane under ordinary conditions. The properties of the semi-permeable membrane with an affixed treating material are such that the treating material is released in a controlled manner either by efflux from micropores (5) or by hydrolysis of a chemical bond (6). Release kinetics are based on the nature of the bond between membrane and treating material, and/or the composition of the microporous layer.

The release of a treating material is unidirectional, as the semipermeable membrane is substantially impermeable to macromolecules. Depending of the composition of the semipermeable membrane, small molecules, e.g., cellular waste products (19), and other small toxins, are free to diffuse through the membrane and away from the tissue or fluid being treated. The novel device consists of a semipermeable membrane with an affixed treating material enclosed within a cartridge (9) that has been modified to be placed either in series or in parallel with extra-corporeal blood or fluid management apparatus. The cartridge, in its principal embodiment, is designed to be labeled by the pharmaceutical supplier, single use and disposable.

The treating material can be any substance of benefit to the tissue or fluid being treated. Examples of potential treating materials include, but are not limited to a growth factor, an anticoagulant, extracellular matrix components, morphogenetic molecules, blood products, proteins, cell stimulating factors, chemotherapeutic agents, diagnostic reagents, antibodies, colony simulating factors, antineoplastic agents, cells, ions, binding molecules, antibiotics, vitamins, cofactors, inorganic catalysts, enzymes, nuclear, ionic or ionizing radiation, free radical scavengers, radiofrequency, electricity, a pharmaceutical, and organic tissue.

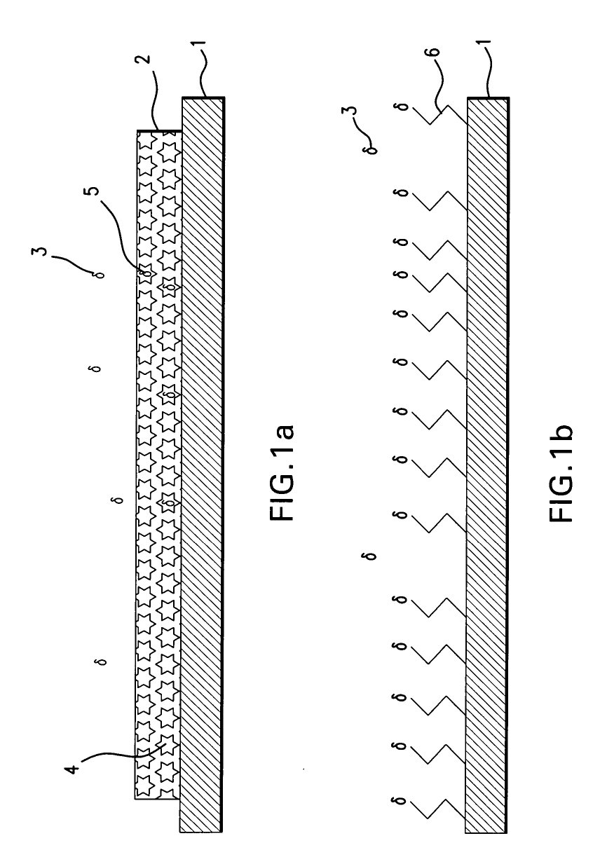

The membrane component of the device and novel method of its use, is equivalent to that previously disclosed in the parent application (Ser. No. 08/557,432) and issued patents (U.S. Pat. No. 5,466,262 and U.S. Pat. No. 5,653,760). The minimally porous, semi-permeable membrane (1), is the same composition that I have described previously. This membrane can be manufactured with any material as long as it has the means to substantially restrict the through passage of a treating material. Suitable examples include but are not limited to Millipore filters, PTFE, and standard dialysis membranes. Typically, the molecules that can freely pass this membrane are on the order of 100 Daltons; however membranes with larger or smaller pore sizes can be used depending on the clinical requirements. In one embodiment, a microporus second layer (2) is affixed to the semi-permeable membrane (1). In this embodiment medication originates within micropores (5) and subsequently diffuses in a directional manner toward the tissue to be treated. Free medicine (3), and an empty micropore (4) are depicted as shown. The rate of efflux is dependent upon the microporus properties of the sheet (2) and the means employed to affix the treating material. In the example shown in FIG. 1b, this means is a chemical bond (6). Permeability characteristics and treating-material release kinetics can also be altered by making the membrane substantially hydrophobic, or hydrophilic. In this way, steroids, hormones or other like treating materials can be delivered. The electric charge across the membrane can also be varied, thereby altering both the permeability characteristics and/or the release kinetics of the treating material.

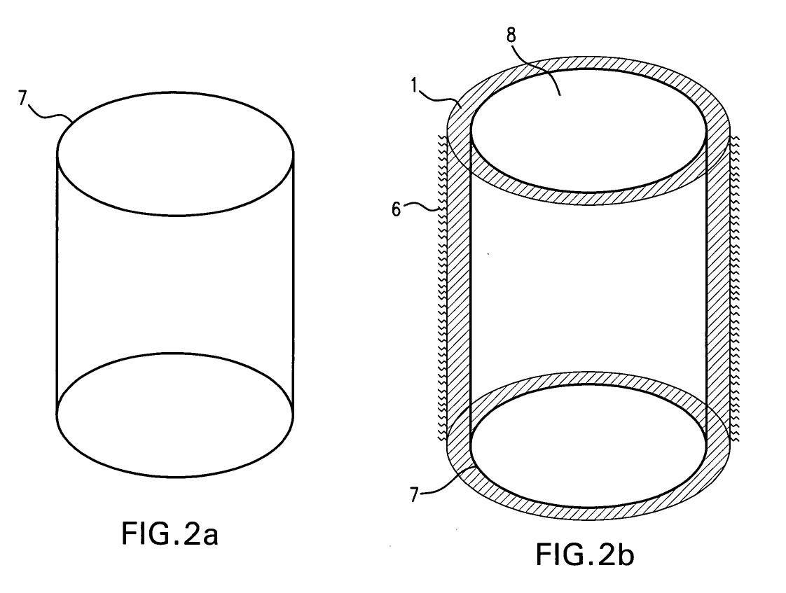

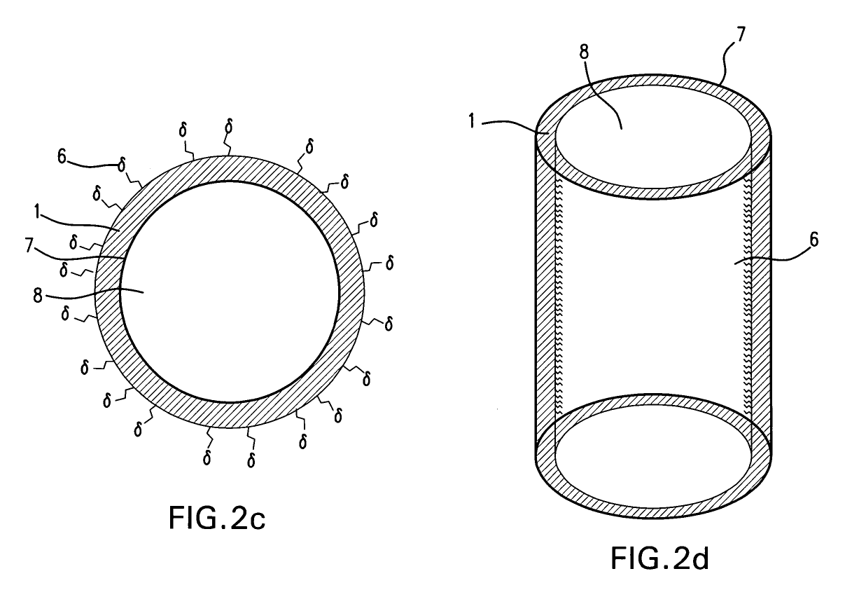

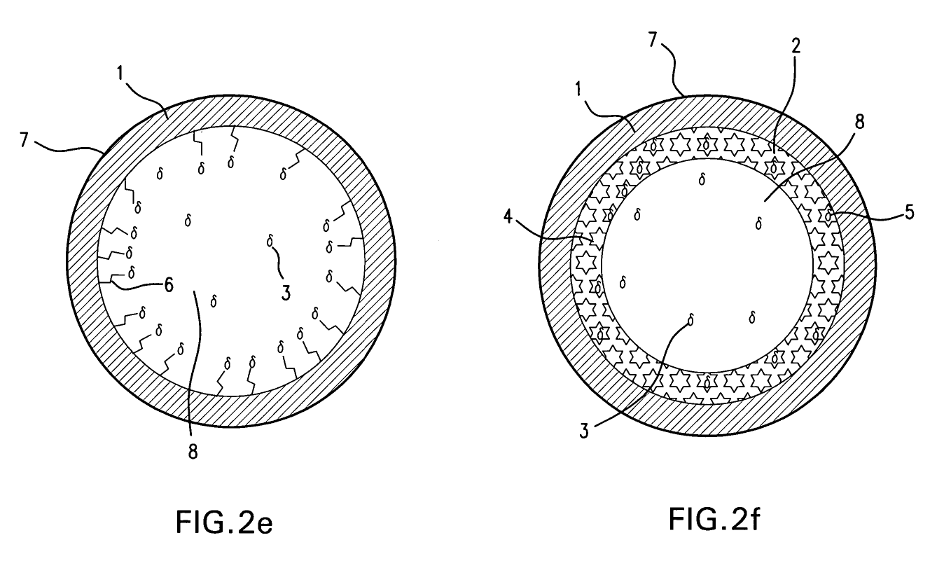

The device of the present invention involves the placement of the above-described semipermeable membrane inside a housing (9). In a principal embodiment this membrane is affixed to a scaffolding (7). The scaffolding, shown if FIG. 2a, is designed to support the membrane as fluid passes over it. Optimally, the scaffolding sufficient to maximum the surface area of the membrane available to the passing fluids. Consequently, the material used to manufacture the scaffolding is not important. Ideally it should be easily sterilized and inexpensive, as the goal is to provide a disposable device.

FIG. 2b demonstrates a semi-permeable membrane (1) affixed to such a scaffold. In this embodiment, the medication-coated surface is directed away from the lumen. This configuration would be useful either to pass blood or other fluid to be treated over the outside of the membrane while a different fluid is passed inside the lumen. In this arrangement, small molecules would be permitted to cross between lumen and outer compartment while medication, cells and plasma are contained in the outside chamber. The fluid within the lumen can be of different temperatures, ionic compositions or osmolality. The membrane itself can be made to have an electric charge or be made to incorporate receptor-binding sites for particular molecules. The only requirement for the treating material membrane arrangement is that the membrane be substantially impermeable to at least one soluble treating material. FIG. 2c represents a cross section of the device illustrated in FIG. 2b.

FIG. 2d represents a membrane scaffold complex with the medication side directed towards the lumen. In this arrangement as fluids pass through the lumen, they come in contact with the treating material affixed to the membrane. In one embodiment the treating material represents antibiotic or other soluble medicine. Depending on the nature of the bond between treating material and membrane, the release kinetics can be manipulated. For example, if it is desired that a treating material be released over the course of an hour, then one simply needs to manufacture the membrane-treating material complex with a bond having a specific rate of release. Esther bonds although not required, for use in this application, can be used for such a purpose. The release kinetics and/or the membrane characteristics can also be altered by application of an external force such as an electric charge. For example, if one applies a positive charge to the membrane at the beginning of a treatment, negatively charged ions will be attracted and bound to the membrane. If one reverses the membrane charge during the treatment, there results in a sudden increase of negatively charged ions in solution.

Another application of the arrangement illustrated in FIG. 2d, is the binding of undesirable molecules or toxins within blood plasma. In this arrangement the membrane would be coated with specific receptors for such molecules and, as the blood passed over the membrane, these toxins would be bound and removed from circulation. When the binding sites are filled up the cartridge could be replaced. In the same manner, cells displaying particular antigens could also be sequestered on the membrane and either disposed of or otherwise utilized simply by removing the cartridge. Moreover, molecules, ions, or cells having a particular surface charge could be induced to marginate on the membrane by varying the electric charge associated with the treating material, the membrane surface, or the membrane itself. Furthermore, if one passes a second fluid of higher osmolatity along the outer surface of the membrane (that surface opposite to the lumen) there results a relative hemoconcentration of macromolecules within the lumen. This increased concentration results in more rapid binding of macromolecules to the membrane's surface.

FIG. 2e demonstrates the arrangement shown in FIG. 2d, shown in cross section. In this diagram, intravenous fluid has been passed into the lumen and some of the affixed treating material is now soluble and exists free in solution. FIG. 2f displays a cross section of a scaffold membrane complex utilizing the malleable fracture stabilization device with micropores. In this illustration, medicine imbedded within the microporus component (5), reconstitutes and becomes free in solution 3). An empty micropore is also shown (4). Note that the medicine can only enter the lumen because the minimally porous properties of the semipermeable membrane (1). Because a treating material can be made to diffuse out of these pores across a concentration gradient, a treating material such as heparin can be repeatedly added to empty pores by placing a very concentrated solution of treating material within the lumen prior to use in a patient.

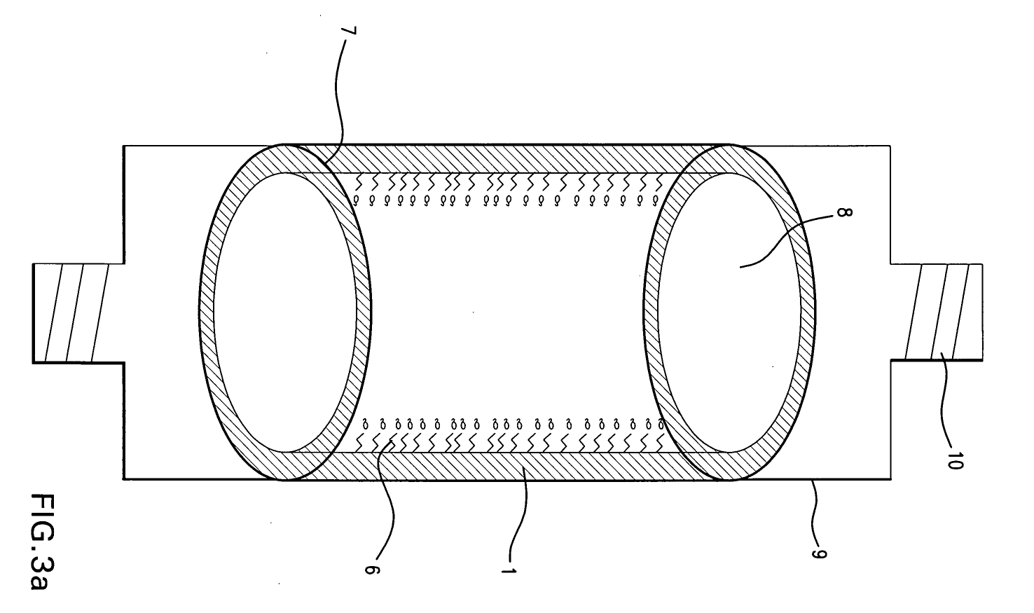

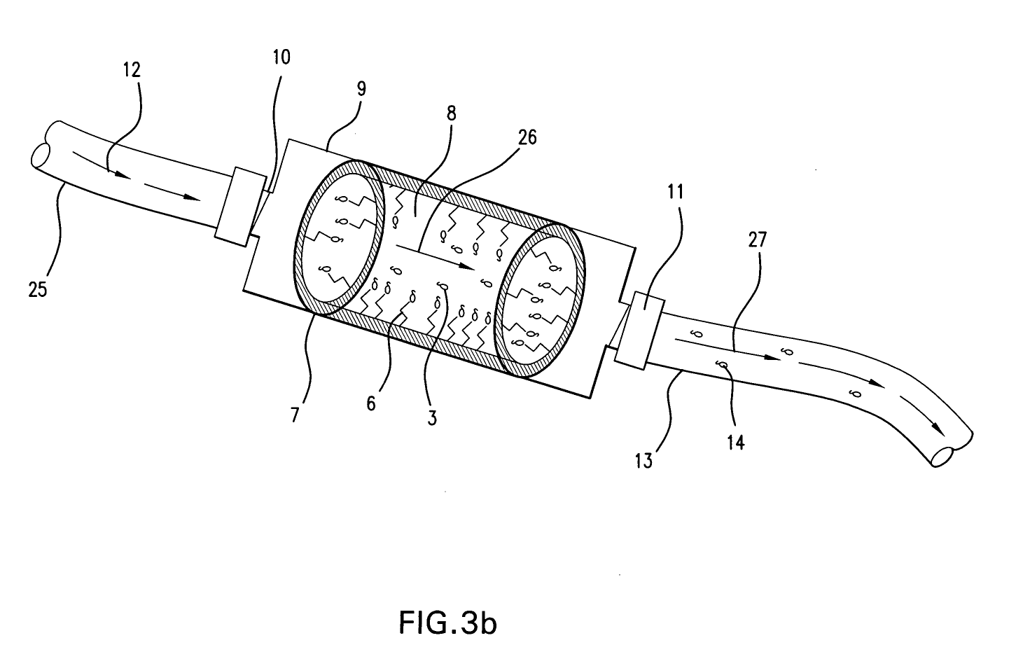

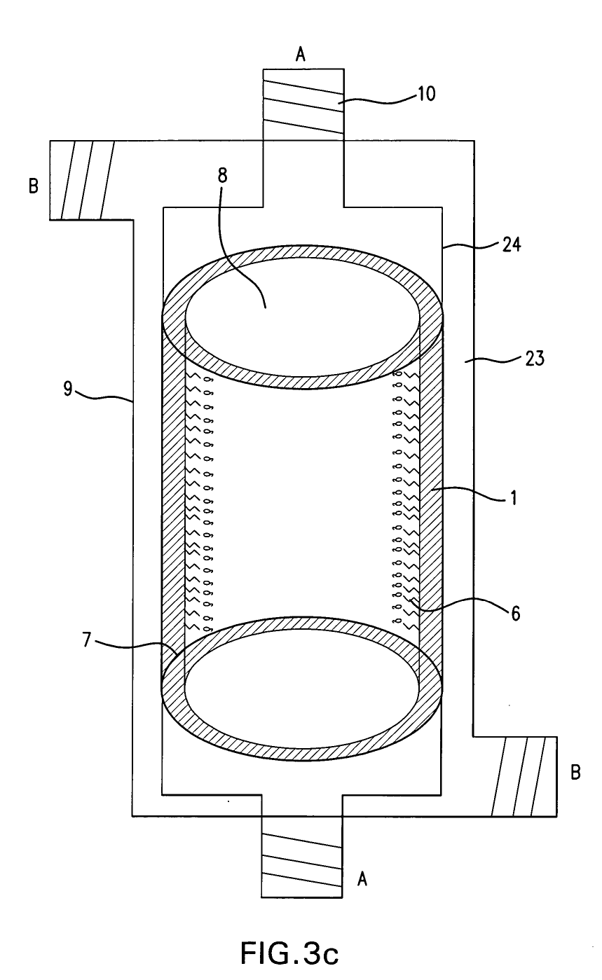

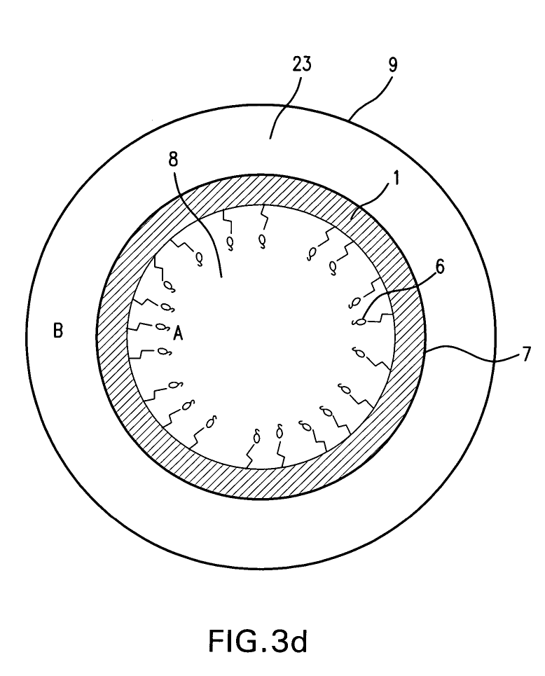

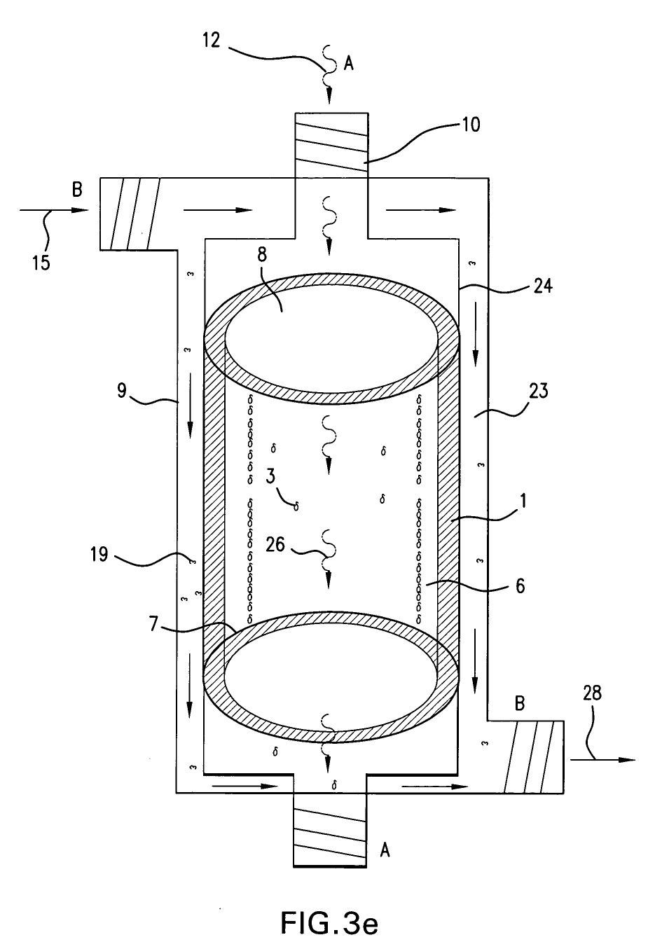

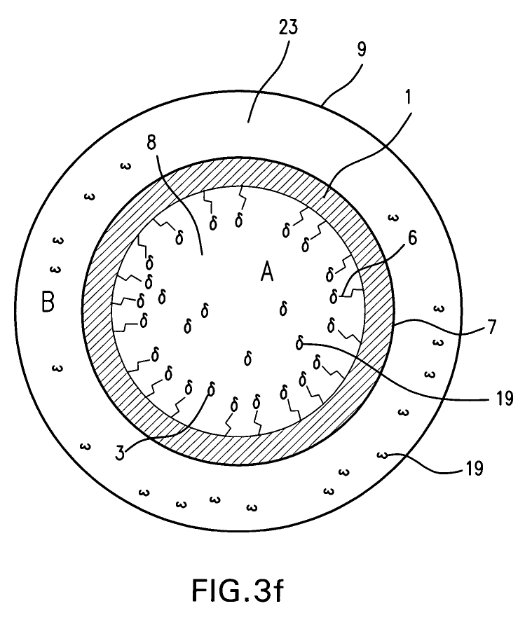

FIG. 3a illustrates how the medication-coated membrane can be housed in a compact cartridge assembly. In this illustration, the inner surface of the semipermeable membrane has been coated with the treating material. The outer non-treated surface of the membrane is intimately associated with both the scaffold and the outer housing. The fluid to be treated is designed to enter one end of the assembly and exit the opposite end. While the fluid is within the chamber, several events can occur depending on the treating material applied to the membrane. If a dry medicine is affixed to the membrane, it becomes reconstituted within the chamber as a fluid passes through. The amount of medicine that is ultimately reconstituted per unit of fluid is dependent on several factors including the rate of fluid transit; the rate of fluid across the membrane, the nature of the bond between treating material and the membrane, and the total concentration of treating material on the membrane.

FIG. 3b illustrates the use of the cartridge assembly in FIG. 3a. The fluid to be treated enters the cartridge via tubing affixed to the cartridge via Luer Lock mechanism. Fluid then enters the cartridge and some of the affixed medicine becomes reconstituted into solution. As the fluid moves through the cartridge and exists via the efferent tubing, soluble treating material has been delivered directly to the fluid. Alternatively, the treating material could be a binding protein that can bind cells, or macromolecules. In this case, a higher concentration of these substances would be present in the fluid entering the cartridge, than in the fluid leaving the cartridge. When these binding sites become substantially occupied, the cartridge can be replaced. Following removal of the cartridge, one can easily elute these molecules or cells for later use.

FIG. 3c illustrates a more complex cartridge arrangement in which there are two interior chambers instead of one. The lumenal chamber (8) is separated from the extra lumenal chamber by a semipermeable membrane to which a treating material has been attached. Small (less than 100 Dalton) molecules are free to cross from the inner to the outer chamber, and vise versa. Macromolecules, however, are not and would be contained within the chamber they originated in. In the illustrated example, the medication-coated surface is directed towards the lumen, however this arrangement may be reversed. In-flow and out-flow ports have been illustrated (FIG. 3c). This two chambered arrangement allows one to take full advantage of the semipermeable membrane by varying the concentration of ions and macromolecules in the chambers. FIG. 3d shows a cross section of FIG. 3c.

FIG. 3e depicts the two-chambered cartridge in use. In this example, two columns of fluid are entering the device. The first column is represented by the wavy arrows and enters from the lumenal entry port (12). The second column of fluid is represented by the straight arrows and enters via the external chamber entry port (15). The macromolecules originating within the lumenal fluid (12) are contained within the lumenal chamber (8) by the semipermeable membrane. The treating material is also contained within the lumenal chamber. If the side of the membrane containing the medication coating is placed facing the external chamber, the treating material remains in the external chamber. Small molecules (19), however, are free to cross the semipermeable membrane in response to changes in fluid composition. The fluid exiting the external chamber “B” (28), is similar to dialysis fluid in that its composition is a reflection of the molecular exchange that has taken place across the semipermeable membrane within the cartridge.

FIG. 3f is a cross section of FIG. 3e. Note that the free treating material is contained within the lumen, while small molecules are free to follow their concentration gradients. The two chambers, “A” and “B”, are separated by the medication-coated semipermeable membrane.

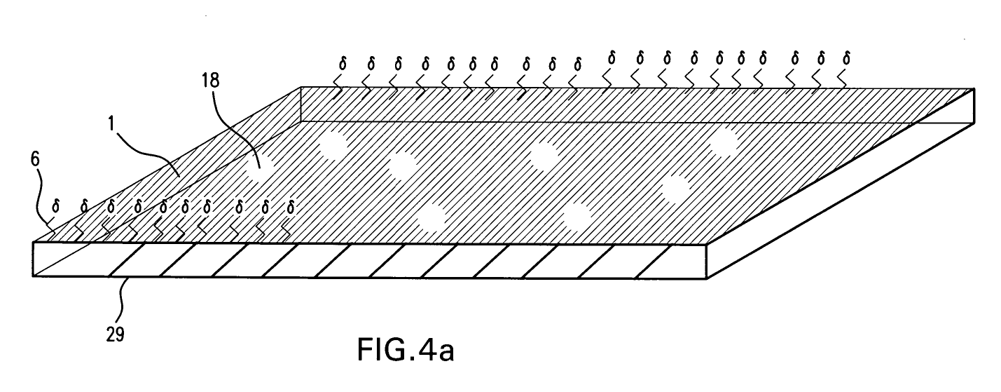

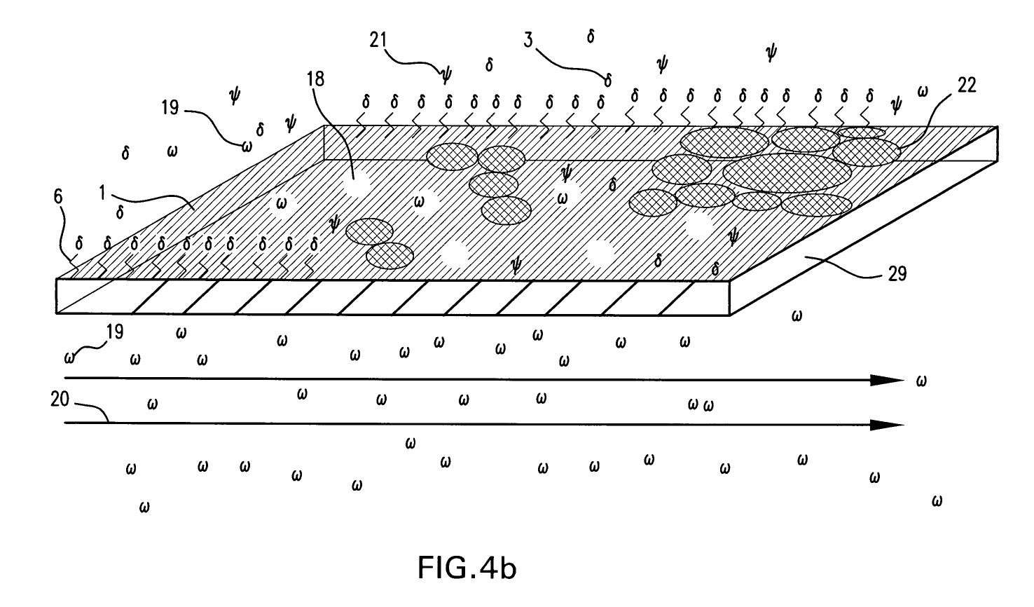

FIG. 4a illustrates an oblique view of the medication coated semi-permeable under which has been placed a very loosely woven supporting scaffold. I have found that the medication coated semi-permeable membrane offers an unusually supportive substrate for cell growth (FIG. 4b). The semipermeable membrane can function as a way to dialyze cellular waste products away from growing cells thereby markedly increasing their rate of growth. The rate of growth can be further accelerated by the affixation of a treating material to the surface onto which the cells grow. The purpose of the supporting scaffold is to elevate the membrane above the floor of a laboratory well or tissue culture plate thereby creating two separate chambers within the dish separated by this semipermeable membrane this embodiment cells would be plated on the medication coated surface and their waste products would be able to diffuse through the membrane into the lower chamber. A wash fluid can then be passed through the inferior chamber such that waste products are swept away. The removal of waste products drives cellular reactions forward, thereby increasing the rate of cell growth. Note that the treating material, and nutrients provided to the cells are not able to diffuse through the membrane, and are concentrated in the solutions surrounding the cells. In a similar fashion, a medication-coated membrane can be used to line the surface of disposable chambers for use in laboratory. Diagnostic evaluations of cells obtained at bone marrow aspiration or flow cytometry could be performed, as reagents could be selectively concentrated or removed, based on the composition of the membrane used. Treatment regimes of cells could also be undertaken in vitro using this system, e.g., in vitro fertilization. Depending on the medicine affixed to the membrane, e.g., a particular cytotoxic drug, clonal selection or other selective cell proliferation treatment could be performed.

### Ramification and Scope

Accordingly, the reader will see that the use of a malleable fracture stabilization device with micropores for directed drug delivery and an apparatus for managing macromolecular distribution can be used in several new and useful ways, distinct from those disclosed in the prior art including pending Ser. No. 557,432 and U.S. Pat. Nos. 5,466,262 and 5,653,760. Furthermore, the reader will note that the present invention addresses several outstanding problems apparent to those working in the art.

Specifically, the present invention is able to bind fluid-borne toxins at the surface of the membrane, thereby minimizing the chance that complications relating to drug toxicity will occur. The invention also teaches the use of a dialysis membrane to which heparin or other anticoagulant has been affixed, thereby substantially preventing thrombosis on the membrane while limiting the amount of heparin that must be given systemically. Furthermore, the present invention provides a new and useful mechanism to deliver a treating material directly into the intravenous line from a pre-labeled vial, at a precise rate, and in a minimum volume of fluid. Remarkably, the invention can also be used to deliver treating materials directly to cells and tissues at a defined rate, while at the same time permitting small metabolites and other small toxins to wash away.

As the reader can appreciate, the device and the method provided is not only a major advance in the extracorporial treatment of blood and other fluids to be infused into a patient's body; but it is also a significant advance in the harvesting of cells and molecules from fluids and blood, in the treatment of cells and fluids in the laboratory, and in the growth of artificial organs such as skin.

Thus the scope of the invention should be determined not only by the content of the above sections and the few examples given, but also by the appended claims and their legal equivalents.

1-8. (canceled)

9. A device to provide the presentation of a treating material to fluids or tissues for use in human or veterinary medicine comprising:
i) a layer of material that is minimally porous to macromolecules, having a first and second major surface, the first major surface being adapted to be placed adjacent to a tissue or fluid to be treated, a second major surface being adapted to be placed opposite to a tissue or fluid to be treated, the layer being capable of releasing at least one treating material in a unidirectional manner, the layer also being capable of restricting the through passage of at least one type of macromolecule there through and
ii) a treating material bound to the first major surface of the layer.

10. The device of claim 9 which is capable of being affixed to a supporting scaffold.

11. The device of claim 9 which is capable of being housed within a cartridge.

12. The device of claim 9 which is capable of being affixed to a microporus layer containing at least one treating material.

13. The device of claim 9 wherein the at least one treating material is selected from the group consisting of a growth factor, an anticoagulant, extracellular matrix components, morphogenetic molecules, blood products, proteins, cell stimulating factors, chemotherapeutic agents, diagnostic reagents, antibodies, colony stimulating factors, antineoplastic agents, cells, ions, binding molecules, antibiotics, vitamins, cofactors, inorganic catalysts, enzymes, nuclear, ionic or ionizing radiation, free radical scavengers, radiofrequency, electricity, a pharmaceutical, and organic tissue.

14-17. (canceled)

18. The device of claim 9, wherein the treating material is a macromolecule.

19. The device of claim 9 wherein the treating material is an antineoplastic agent.

20. The device of claim 9 wherein the treating material is an antibiotic.

21. The device of claim 9, wherein the device is capable of preferentially delivering treating material to tissue on one side of the layer.

22. The device of claim 9, wherein the layer comprises a porous polymeric matrix.

23. The device of claim 9, wherein the layer is semipermeable to the treating material.

24. The device of claim 23, wherein the treating material is capable of diffusing out of pores in the semipermeable layer according to a concentration gradient.

25. The device of claim 9, wherein the treating material is capable of forming a coating on one side of the layer.

26. The device of claim 9, wherein the layer comprises a hydrophobic polymer.

27. The device of claim 9, wherein the treating material is attached to the first major surface of the layer by hydrophobic forces.

28. The device of claim 9, wherein the treating material has a charge that interacts with the layer to substantially restrain the treating material from being washed away.

29. The device of claim 9, wherein the device is capable of delivering treating material directly to cells or tissue while permitting small metabolites and toxins to wash away.

30. The device of claim 9, wherein the device is capable of substantially preventing the treating material from being released into solution.

31. The device of claim 9, wherein the layer comprises a substantially hydrophobic polymer coating on a support scaffold and is capable of having a treating material affixed to its surface for delivery directly to cells or tissue.

32. The device of claim 9, wherein the device is capable of permitting small metabolites to wash away.

33. The device of claim 9, wherein the device is capable of preferentially directing the treating material to cells or tissues to be treated.

34. The device of claim 9 wherein the device is capable of substantially containing the treating material next to cells or tissue in need of treatment.

35. The device according to claim 9 further comprising means to substantially direct one surface of the layer toward tissues or fluids to be treated.
